(19) United States
(12) Patent Application Publication
(10) Pub. No.: US 2016/0008628 A1
(43) Pub. Date: Jan. 14, 2016

(54) Novel Treatment Methodologies Using Light Therapy

(71) Applicants: Larry Dwayne Morries, Lakewood, CO (US); Theodore Allen Henderson, Centennial, CO (US)

(72) Inventors: Larry Dwayne Morries, Lakewood, CO (US); Theodore Allen Henderson, Centennial, CO (US)

(21) Appl. No.: 14/797,067

(22) Filed: Jul. 10, 2015

Related U.S. Application Data
(60) Provisional application No. 62/023,150, filed on Jul. 10, 2014.
(60) Provisional application No. 62/024,770, filed on Jul. 15, 2014.
(60) Provisional application No. 62/024,795, filed on Jul. 15, 2014.

(51) Int. Cl. A61N 5/06

(52) U.S. Cl. CPC A61N 5/0622; A61N 5/0618; A61N 2005/067

(57) ABSTRACT

An exemplary method of treatment involves the non-invasive delivery of near infrared light (NIR) in 600-1200 nm wavelengths at surface wattage of 10.01-20.00 watts applied using a continuous motion technique. Treatment duration can be 30-1800 seconds, and can be conducted using a specific pulsed NIR technique for minimizing surface tissue heating. This is a novel process of non-invasive NIR/laser therapy. Prior applications used low levels of NIR therapy—most less than 1 watt with some up to 5 watts. Embodiments of the methodologies can be practiced with a laser unit having control for wavelength and for 1-20 watts. A Pulsed (on/off laser beam) control for 1-1000 milliseconds may also be utilized. An applicator with 1-2 cm aperture can be used to produce approximately 0.64-1.95 Joules per cm2 fluence in the depth of tissue. A display detailing watts, time, continuous or pulsing, and Joules of therapy is useful.

### CROSS-REFERENCE TO RELATED APPLICATIONS

This application claims the benefit of U.S. Provisional Application No. 62/023,150 and filed on Jul. 10, 2014, as well as U.S. Provisional Application No. 62/024,770 and filed on Jul. 15, 2014, as well as U.S. Provisional Application No. 62/024,795 also filed on Jul. 15, 2014, all of which are specifically incorporated by reference herein for all that they disclose and teach.

### TECHNICAL FIELD

The present invention relates generally to injury, disease, and dysfunction of the brain, spinal cord and/or nerves; neurodegeneration; neuro-muscular injuries and diseases; and/or orthopedic and sports related injuries; and more particularly, to novel treatment methodologies using light therapy to address these injuries, diseases and/or dysfunctions.

### BACKGROUND

The novel treatment methodologies of the present invention can be utilized to treat a large number of health issues, including, but not limited to: Traumatic Brain Injury (TBI); Idiopathic Parkinson's Disease (IPD); Alzheimer's Disease, Senility, and Dementia (ADSD); Post-Traumatic Stress Disorder (PTSD); Depression and Anxiety (DA); Multiple sclerosis (MS); and Spinal Cord Injuries (SCI), stroke (STR), neuropathy/polyneuropathy (PNL), radiculopathy/radiculitis (RDL), and mitochondrial dysfunction (MTD).

Traumatic Brain Injury

The Centers for Disease Control and Prevention estimated that 1.5 million Americans sustained TBI annually in 2000. As of 2006, the estimates had risen to 1.7 million brain injuries annually. Undoubtedly, these point prevalence proportions will increase as military personnel return home and the problem of repeated mild TBI (mTBI) becomes more recognized in sports. Current estimates of the prevalence of TBI among Veterans range from 9.6% to 20%, with an estimated total of more than 300,000 cases of TBI among military personnel since 2000. The current estimates of the combined number of sports-related concussions and brain injuries in the United States are 1.6-3.8 million annually.

TBI results in a wide spectrum of neurological, psychiatric, cognitive, and emotional consequences. In part, the variation is related to the severity of the injury (mild, moderate, severe TBI), which are stratified based on Glascow Coma score, periods of unconsciousness, and degrees of amnesia. Furthermore, the diversity of sequalae can be related to the areas of the brain that are injured, the severity of the injury (highly variable within the classification of “mild” and “moderate”), and the evolution of the injury over time due to neuro-inflammatory processes. Additional mechanisms thought to underlie the damage of TBI include decreased mitochondrial function, calcium and magnesium dysregulation, excitotoxicity, disruption of neural networks, free radical-induced damage, excessive nitric oxide, ischemia, and damage to the blood brain barrier. Together, these can contribute to a progression of the damage over time.

Patients with TBI can experience: headaches, visual disturbances, dizziness, cognitive impairment, loss of executive skills, memory impairment, fatigue, impulsivity, impaired judgment, emotional outbursts, anxiety, and depression. The situation can be further clouded by secondary and/or comorbid post-traumatic stress disorder (PTSD), depression, and anxiety, which can have symptoms that overlap with those described above and appear to be increasingly likely with repetitive concussive or subconcussive brain injury.

Idiopathic Parkinson's Disease

Idiopathic Parkinson's disease (IPD) is a progressive neurological disorder characterized by selective degeneration of dopaminergic neurons in the substantia nigra. Manifestations of clinical symptoms often do not occur until at least 60% of substantia nigra neurons are lost and/or dopaminergic integrity is reduced significantly. While IPD may be the most prevalent form of parkinsonism, it shares, in part, symptoms with progressive supranuclear palsy, multiple system atrophy (MSA), vascular parkinsonism, and dementia with Lewy bodies. Symptoms of tremor, bradykinesia, postural instability, rigidity, and autonomic dysfunction can overlap, to a greater or lesser degree, in each of these disorders. Specific clinical symptoms, such as cerebellar signs in MSA, gaze palsy in progressive supranuclear palsy, or overt dementia and visual hallucinations in dementia with Lewy bodies, may only manifest at a later stage of the disease. Given the multifarious presentation of IPD, the discrimination of these diseases early in the course of illness can be challenging. Yet, the correct diagnosis is critical, as each disease process has a different pathology, different progression, and different response to medication.

Alzheimer's Disease, Senility, and Dementia

Between 1997 and 2025, the number of individuals worldwide over 65 years of age will increase from 381 million to 823 million. Age-related conditions can be expected to increase in frequency as the population ages. In particular, the dementias, such as Alzheimer's disease (AD), frontotemporal dementia (FTD), dementia with Lewy bodies (DLB), vascular dementia (VaD), and other stroke-related disorders, will increase in frequency, and those afflicted will place increasing demands on medical systems and on families. The frequency of dementias doubles with every 5 years of age over 60. Without effective treatment, the number of persons with dementing illnesses will quadruple in the next 50 years. Worldwide, an estimated 81 million people will have some form of dementia. The number of people with AD in the United States alone, currently approximately 5 million, is expected to exceed 13 million by 2050. In addition, 19% of those over the age of 65 years develop mild cognitive impairment (MCI) which also is referred to as senility or early dementia. It is a potential precursor to a dementia. It is estimated that approximately 156 million persons worldwide will be affected by MCI. Furthermore, new medications in development—some of which will target amyloid accumulations, such as gamma and b secretase inhibitors/modulators, as well as alpha secretase activators and tau kinase inhibitors—are relatively unlikely to clear a large burden of amyloid such as is found in late disease and even less likely to reverse the pathology when secondary events (such as inflammation) have occurred. Indeed, in August 2010, Eli Lilly halted clinical trials of a gamma secretase inhibitor due to lack of effect on cognitive function. While this pharmaceutical was apparently effective in reducing amyloid deposits in the form of plaques, subjects showed marked worsening of cognitive function. Currently, there are no effective treatments for Alzheimer's disease or the other dementias. Only mitigative and palliative care can be offered.

Post-Traumatic Stress Disorder

Post-traumatic stress disorder (PTSD) is a mental health condition which is triggered by either experiencing or witnessing a terrifying event. Recent evidence has shown that certain genes increase the vulnerability to PTSD. Symptoms may include nightmares, intrusive thoughts about the traumatic event, severe anxiety, and episodes of reliving the traumatic event (flashbacks). Symptoms may start within three months of an event, but also may not appear until years after the event. In addition to symptoms of intrusive memories, efforts to avoid people, places and things that are reminders of the event, irritability, emotional numbness, loss in interest and pleasure in usual activities, social withdrawal, mood change, memory problems, relationship problems, guilt and shame are common symptoms.

Depression

Depression is a profound problem in American and worldwide. Depression is more than “having a bad day” or “feeling blue”. It is a long-lasting experience of low mood, loss of enjoyment in life, loss of interest, low energy, changes in sleep and/or appetite, and a decrease in one's ability to think clearly (cognition). Many people with depression experience extreme distress and anguish. Some feel suicidal and may act on those impulses. Depression is found in every country of the world and in every socioeconomic class. The rate of depression worldwide and in America is about 5-7%.

Anxiety

Anxiety is an exaggeration of the normal mental and physical sensations of fear. Anxiety can manifest as fears, nervousness, restlessness, worries, uncertainty, irritability, and trouble concentrating. In children, anxiety can be confused with ADHD, because of restlessness and trouble focusing. Anxiety also can produce physical symptoms, such as trembling, upset stomach, nausea, diarrhea, headache, chest pain, trouble breathing, irregular heartbeats, muscle tension, and sleep disturbance. These physical symptoms are particularly likely during severe anxiety or panic attacks.

Actually, anxiety is a general term for several disorders that produce fear, apprehension, or worry. This includes Social Anxiety Disorder, Panic Disorder, Obsessive Compulsive Disorder, Separation Anxiety Disorder, Post-Traumatic Stress Disorder, and Generalized Anxiety Disorder. Approximately 27% of Americans suffer from one form of anxiety or another.

Research has shown that abnormal levels of certain neurotransmitters are present in the brain of people with anxiety disorders. Treatments for anxiety disorders include medications, a variety of therapy techniques, physical exercise, and even certain supplements. Some medications for anxiety, such as the benzodiazepines, are addictive and interfere with learning and memory function. Other medications which are often prescribed for anxiety, the SSRI's, can actually worsen anxiety in some patients. No single medication seems to be effective for all patients with anxiety disorders. Many patients with anxiety find only partial relief with currently available medications.

Multiple Sclerosis

Multiple sclerosis (MS) is a neurological disease in which elements of the immune system attacks the protective sheath (myelin) that covers axons in the spinal cord and brain. Ultimately, the nerves themselves may degenerate and die. Signs and symptoms vary widely, depending on where in the central nervous system the damage occurs, the amount of damage, and which nerves are affected. Symptoms can include impaired motor function, alertness, cognitive function, or emotional control. There's no cure for multiple sclerosis. However, current treatments can help quicken recovery from attacks, modify the course of the disease, and mitigate or palliate symptoms.

Spinal Cord Injury

A spinal cord injury signifies damage to any part of the spinal cord or nerves emerging from the spinal canal. This injury is largely irreversible with currently available treatments. The injury often causes permanent changes in movement, strength, sensation and other body functions. Injury to the spinal cord can be complete—involving all feeling and motor control below the level of the injury—or partial—leaving some sensation or motor control below the lesion.

Depending on the level of the injury, spinal cord injuries may result in one or more signs and symptoms, such as loss of movement, loss of sensation, including the ability to feel heat, cold and touch, loss of bowel or bladder control, exaggerated reflexes, pain or an intense stinging sensations, and/or difficulty breathing, coughing or clearing secretions from the lungs.

Mild-to-Moderate Stroke (STR)

Stroke is the result of blockage or ischemia of the blood vessels to the brain. Stroke can vary from minor with only transient clinical impact to fatally severe. Mild-to-moderate stroke usually results in loss of some motor, sensory, and/or executive function accompanied by neurodegenerative changes. Currently, the treatment of stroke is limited to the acute setting within hours of the event or to physical rehabilitation. Treatments which improve brain function after stroke are sorely needed.

Neuropathy/Polyneuropathy (PNL)

Neuropathy is a dysfunction of nerve transmission/nerve conduction in the peripheral nervous (usually the lower extremities). The most common cause is diabetes, but there are other etiological factors such as carpal tunnel compression, spinal disc herneation, etc. Neuropathy can involve one or multiple nerves (polyneuropathy). It is generally mild-to-moderately disabling and/or can contribute to Restless Leg Syndrome. Currently, the treatment for neuropathy is prevention and then the pharmaceuticals, gabapentin or pregabalin, to treat the uncomfortable symptoms of numbness, tingling and pain. Note: these pharmaceuticals do nothing to restore the function of the nerve. We have shown that infrared light improves nerve conduction (i.e., improve function of the nerve) and reduces symptoms.

Radiculopathy/Radiculitis (RDL)

Radiculopathy is irritation to spinal cord nerve roots usually caused by some sort of direct traumatic event. Radiculitis results from irritation to the sensory nerve root resulting in pain. These can occur with almost any traumatic event, intervertebral disc disease, spinal compression, spinal stenosis, and other degenerative processes. Currently, treatment is limited to surgical intervention, spinal manipulation, pain medications, injections of steroids or opiates, physical therapy, and traction. None of these treatments, in and of themselves, restore nerve function, although surgical intervention can relieve pressure on the nerve; however, surgery has a recurrence rate of up to 50% over 5 years.

Mitochondrial Dysfunction (MTD)

Mitochondrial dysfunction leads to a decrease in adenine triphosphate (ATP) production and loss of regulation of genetic, growth, and neuronal support functions Infrared light has been shown to stimulate many of the essential mitochondrial functions.

In the past, a number of low level laser therapies have been developed that attempt to address one or more of the above issues. Near-infrared (NIR) light has been investigated for its ability to modulate intracellular reparative mechanisms. NIR can facilitate wound healing, promote muscle repair, and angiogenesis. The application of NIR by low-power laser or by light-emitting diode, referred to as either laser phototherapy or near-infrared photobiomodulation, has been studied and applied clinically in a wide array of ailments, including skin ulcers, osteoarthritis, peripheral nerve injury, low back pain, myocardial infarction, and stem cell induction. Since NIR passes relatively efficiently through bone, several studies of transcranial near-infrared light therapy (NILT) in animal models of brain damage have been conducted by multiple laboratories. A large clinical trial of NILT for acute stroke showed clinical improvement; however, a subsequent Phase III clinical trial failed to show benefit at an interim futility analysis. This and other findings of ineffective protocols for NIR in a variety of pathological conditions raise an important question about the necessary elements of an effective therapy.

Current data indicates that near-infrared photons of wavelengths between 600-1,200 nm are critical to infrared phototherapy. These wavelengths are absorbed by cytochrome-c-oxidase in the mitochondrial respiratory chain which increases the activity of the respiratory chain and leads to an increase in adenosine triphosphate (ATP) production. Simply increasing ATP in wounded or underperfused cells may be sufficient to stimulate cells in areas of injury or metabolic derangement; however, tissue culture and animal studies implicate secondary molecular and cellular events. Near infrared light appears to alter nitric oxide levels, which may have downstream effects. NIR appears to modulate reactive oxygen and reactive nitrogen species. As a result of the primary events in the mitochondria there is increased energy in the cell. There is a resulting increased oxygenation and displacement of nitric oxide within the cell. Early response genes are activated. Near-infrared light activates nuclear factor kappa B, which is a redox sensitive transcription factor. This pro-survival transcription factor modulates the expression of numerous genes, including ones involved in inflammation, early response, and cell survival. As a consequence of near-infrared phototherapy numerous genes in both the mitochondria and the cell nucleus are activated. There is increased transcription of over 100 genes. In particular, cell survival genes, and neural differentiation factors are transcribed. Increased synaptogenesis occurs. The production of numerous growth factors increases; and several inflammatory mediators are upregulated.

However, despite the benefits of NIR, current low-level NIR treatments fail to penetrate to sufficient depths while retaining sufficient remaining energies. Our recent tissue studies demonstrate no penetration of low-level NIR energy through 2 mm of skin or 3 cm of skull and brain. Therefore Low Level Infrared Therapy is not effective for treatment of Neuro-Degenerative conditions. Furthermore, it does not penetrate down to the joints of muscular tissue in a high percentage of most sports related injuries, falls and other neuro-musculature conditions.

Conversely, our treatment methodologies, using medium-level 10.01-20.00 watt (continuous or pulsed), yields 0.45% to 2.9% of 810 nm light penetration through 3 cm of tissue. A 15 W 810 nm device NIR delivered 2.9% of the surface power density. Pulsing at 10 Hz reduced the dose of light delivered to the surface by 50%, but 2.4% of the surface energy reached the depth of 3 cm. Approximately 1.22% of the energy of 980 nm light at 10-15 watts penetrated to 3 cm. These data are reviewed in the context of literature on low-power NIR penetration, wherein less than half of 1% of the surface energy could reach a depth of 1 cm. NIR in the power range of 10-15 watts at about 810 nm through about 980 nm can provide fluence within the range shown to be biologically beneficial at 3 cm depth. Insufficient energy has been utilized in transcranial infrared treatment protocols. This is a short-coming of previous work and patents in this field. We have demonstrated in our clinical studies and tissue penetration research, the increased penetration of medium-level NIR into these tissues, which results in dramatically improved therapeutic outcome in patients with Depression, Traumatic Brain Injury, Anxiety, and other neurological/psychiatric issues, including those related to neuro-degenerative diseases.

What are needed are novel treatment methodologies that can be utilized to treat TBI, IPD, ADSD, PTSD, DA, MS, SCI, PNL, RDL, STR, MTD and other diseases, conditions, and dysfunctions using medium-level NIR energies.

NIR light in the wavelength range of 600-1,200 nm has significant photobiomodulation capability. Current data most strongly support that absorption of NIR photons by cytochrome c oxidase (COX) in the mitochondrial respiratory chain is the key initiating event in photobiomodulation. COX is a large transmembrane protein of the inner mitochondrial membrane. It contains two copper (Cu) centers and two heme-iron centers. These metal centers have different light absorption peaks. Reduction of CuA occurs with 620 nm, oxidation of CuA occurs with 825 nm, reduction of CuB occurs with 760 nm, and oxidation of CuB occurs at 680 nm. These peaks correspond to the “optical window” associated with the biological effects of NIR. Irradiation of COX increases the activity of the entire electron transport chain producing more adenosine triphosphate (ATP). In addition, COX is auto-inducible and its gene expression is activity dependent, such that NIR irradiation may increase the amount of available COX over time. NIR's effect has been studied in isolated mitochondria preparations. Irradiation with 632 nm light results in increased proton electrochemical potential and increased ATP production. COX activity and oxygen consumption also increases. A more recent study confirmed increased oxygen consumption and showed increased activation of several electron transport chain components. In the setting of acute neurotrauma, this increase in energy supply may be sufficient to reduce the consequences of injury. Neurons are often forced into anaerobic metabolism, which results in acidosis, insufficient energy to maintain ion pumps, and calcium overload. Later in the sequence of events following neurotrauma, more energy is required than at baseline due to the large energy requirements of repair. Increasing ATP during acute neurotrauma alone may be sufficient, but NIR appears to initiate a number of other events in the mitochondria.

With NIR exposure, induction of mitochondrial RNA synthesis, as well as protein synthesis, occurs. More recent work has shown NIR induces the upregulation and downregulation of numerous genes both in the nucleus and in the mitochondria of various cell types. Apoptosis-inhibiting genes are upregulated (eg, Janus kinase binding protein). Meanwhile, apoptosis-promoting genes, such as heat shock 70 k Da protein 1A and caspase 6, are downregulated. Expression of genes for antioxidants and inhibitors of the effects of ROS are increased. Similar work in the retina has shown that genes involved in cell survival, antioxidant production, transcription, and growth factor production are also upregulated in neural retina cells.

Data from tissue culture and animal studies of NIR reveal an increase in growth factor expression and subsequent cell proliferation. Examples of these key growth factors include nerve growth factor, brain-derived neurotrophic factor, transforming growth factor-beta, and vascular endothelial growth factor, which may contribute to late brain remodeling after TBI. For example, a fivefold increase in nerve growth factor mRNA transcription occurred after irradiation of skeletal muscle cell culture with 633 nm NIR light.

Recent data suggest that transcranial NIR phototherapy can increase the process of neurogenesis in adult mice with stroke or TBI. Increased numbers of neuroprogenitor cells have been demonstrated in both the dentate gyms of the hippocampus and in the subventricular zone of the lateral ventricle of mouse models. These cells also demonstrate increased expression of a microtubule protein associated with migrating neuroblasts. Some studies provide evidence that NIR phototherapy may increase the process of synaptogenesis. Together, these processes may aid in the neuroplasticity responsible for neural repair and improved function in cases of chronic TBI.

These cellular changes appear to persist for considerably longer than the interval of light application (when delivered at appropriate wavelengths and amplitudes). For example, low level (red and) near-infrared light therapy (LLLT) of a power density of 0.9-36 J/cm² applied in a single treatment at 24 hours post-stroke in animal models yielded a reduction in neurological deficits, as well as histochemical evidence of neuron proliferation and migration. A single application of LLLT in rodent models of TBI had similar benefits. Interestingly, these benefits were not immediately apparent. Rather, a delay of 1-4 weeks was noted, consistent with a progressive regeneration cascade set in motion by the NIR exposure.

Light has fundamental physical properties which are relevant to its clinical use. Light is a form of electromagnetic radiation which has properties of both waves and particles. Light is characterized by its wavelength (distance between two peaks), frequency, and amplitude. Light is also characterized by its energy content. This energy is quantified as joules (J). The amount of energy delivered per unit time constitutes the power of light in watts (W=J/second). For medical applications, light is typically reported in terms of wavelength (nm), energy (J), irradiance or power density (W/cm²), and radiant exposure or fluence or dose (J/cm²).

NIR has a number of biological effects, but it is critical to understand the physical interactions between tissue and light. When light impinges on the surface, a portion (10%) is reflected. The energy that does penetrate the surface is refracted or bent toward a line perpendicular to the surface. This results from the particle property of light. These particles are, of course, photons. The photons entering the tissue can be transmitted through the tissue, scattered, or absorbed. Scatter increases the volume of tissue impacted by the light. Photons can change direction without loss of energy. Scattering is particularly likely at interfaces between different tissues. Reflection or refraction also occurs at such interfaces. These effects contribute to shortening the distance to which light will travel or penetrate into the tissue.

Most tissues have the capacity to absorb light energy. Usually this is mediated by a molecule absorbing a photon. Molecules containing metal ions have a strong capacity for absorbing photonic energy, but DNA and water also can. The absorption of energy can induce a change in the confirmation and/or function of the molecule.

Penetration of NIR through tissues is determined by several factors: wavelength, energy, attenuation coefficient (composed of scatter, refraction, and absorption), area of irradiance, coherence, and pulsing. In general, longer wavelengths (up to 1,000 nm) will penetrate deeper; however, the absorption of water begins to predominate above 1,000 nm. Increases in power density, in general, will lead to greater penetration. More photons will traverse the tissue. The area of surface irradiation also affects penetration due to scattering effects.

Coherence is a property of light waves in which waves of monochromatic light are aligned such that any point on the wave has the same amplitude and position as the equivalent point in an adjacent wave. Temporal coherence reflects the slight variations in the waveform over time. The more consistent the waveform is, the higher the temporal coherence. Monochromatic light typically has high temporal coherence. Spatial coherence results from the divergence of the light from the point of emission. Laser has virtually no spatial divergence and creates a long narrow volume of coherent light. LEDs are not monochromatic, but emit light in a relatively narrow band over the peak wavelength. LEDs also have significant spatial divergence and therefore a wide volume of space is radiated; however, within that volume only a very small volume contains coherent light. The result is that non-coherent LED sources likely only provide coherent light in a thin volume, usually at surfaces. In contrast, laser generates a long narrow volume of coherent light which can penetrate deeper into tissues.

When coherent light enters a tissue, slight distortions in the timing and the shape of the waves occur. As a result, interference can occur between the waves. Polarization, the angle at which a wave is vibrating, also contributes to interference. On a single wave basis, interference results when the amplitude of the wave at a given point is different from that of an adjacent wave and of the population of coherent light waves. At the point of difference, the amplitudes can either cancel each other out {[+x]+[−x]}, be additive {[+x]+[+x]}, or any variation in between {[+x]+[−y] }. The result of these interactions is a field of randomly distributed points of increased and decreased light intensity, referred to as a speckle intensity pattern. Speckling can have a significant impact on the effective penetration depth. As such, areas of high intensity will penetrate further or will have two to three orders of magnitude greater energy at a given depth.

Pulsing of NIR also increases the depth of penetration and the amount of energy delivered to any given point at the peak of a pulse. Yet, pulsing allows for troughs of energy output such that the overall energy delivered to the tissue can be equivalent or even lower than that delivered by a continuous emission. Pulsing is a property of lasers which cannot be duplicated by LEDs.

We have been utilizing relatively high power (10-15 W) lasers at the wavelengths of 810 and 980 nm in clinic to treat TBI with positive results. NIR as employed in our novel treatment methodologies can also be used in the treatment of stroke. Skin is the first tissue encountered in the clinical application of NIR phototherapy and represents a barrier to effective penetration due to several factors. Human skin has multiple layers and, therefore, multiple interfaces. Each interface is a surface for scatter. In addition, each layer has different inherent optical properties. The epidermis, comprised in part of keratin, collagen, lipids, and melanin, has high absorption in the ultraviolet range, but also absorbs light in the infrared range of 600-1,100 nm. The dermis, comprised in part of collagen, elastin, and proteoglycans, is of variable thickness and penetration varies as a result. Scattering is a predominate property of the dermis. The dermis is also dense with blood vessels and the hemoglobin-rich blood therein. While hemoglobin has absorption peaks at 450, 550, and 600 nm, it also absorbs photonic energy in the clinical NIR range of 800-1,100 nm. The NIR absorption of hemoglobin depends upon its oxygenation status, with carboxyhemoglobin having greater NIR absorption. Altogether, NIR photonic energy must first overcome the hurdle of penetrating the skin to have an impact on deeper structures.

### SUMMARY

One embodiment of a method of treatment is the non-invasive delivery of infrared light in wavelengths between 600-1200 nm at a surface wattage of between 10.01 to 20.00 watts. The duration of each treatment varies, in one embodiment from 30 seconds to 30 minutes, and can be conducted using a specific pulsed NIR technique for minimizing surface tissue heating. This is a novel process of non-invasive NIR/laser peripheral therapy and transcranial phototherapy. Prior applications were with very low levels of near infra-red (NIR) therapy; most less than 1 watt with some up to 5 watts. The therapy disclosed herein is with 10.1-20 watts. Embodiments of the methodologies can be practiced with a laser unit having a control for wavelength, wattage of 1-20 watts and a Hertz control. A Pulsed (on and off laser beam) control for 1-1000 milliseconds may also be utilized. From our data, we estimate that in our clinical applications of high-powered NIR lasers, we are delivering 0.64-1.95 Joules per square cm to a depth of 30 mm. This is 100-fold greater fluence than that delivered by an LED system, but within the range of fluence shown to have beneficial biological effects. A hand held applicator with 1 cm aperture is used in one embodiment to produce approximately 0.64-1.95 Joules per square cm. A display detailing watts, Time, Continuous Wave or Pulsing, and Joules of therapy is useful. The utilized laser unit has FDA approval for use on humans, with safety training, and patient safety measures, eye protection and contraindications.

By using our protocols, we are able to penetrate 3+ cm into the skin/skull/brain. Laboratory research has shown that infrared light can stimulate many brain processes, stimulate the mitochondria, increase ATP, alter nitric oxide (NO) levels, activate genes, increase nerve growth factors, increase synapse formation, and activate other processes.

### BRIEF DESCRIPTION OF THE DRAWINGS

FIG. 1 illustrates an exemplary embodiment of a laser treatment system that can be utilized with one or more novel treatment methodologies for light therapy; and

FIG. 2 illustrates a top plan view of exemplary operations of an embodiment of a novel treatment methodology using light therapy.

### DETAILED DESCRIPTION

In the following discussion, numerous specific details are set forth to provide a thorough understanding of the present disclosure. However, those skilled in the art will appreciate that embodiments may be practiced without such specific details. Furthermore, lists and/or examples are often provided and should be interpreted as exemplary only and in no way limiting embodiments to only those examples.

Exemplary embodiments are described below and in the accompanying Figures. The following detailed description provides a comprehensive review of the drawing Figures in order to provide a thorough understanding of, and an enabling description for, these embodiments. One having ordinary skill in the art will understand that in some cases well-known structures and functions have not been shown or described in detail to avoid unnecessarily obscuring the description of the embodiments.

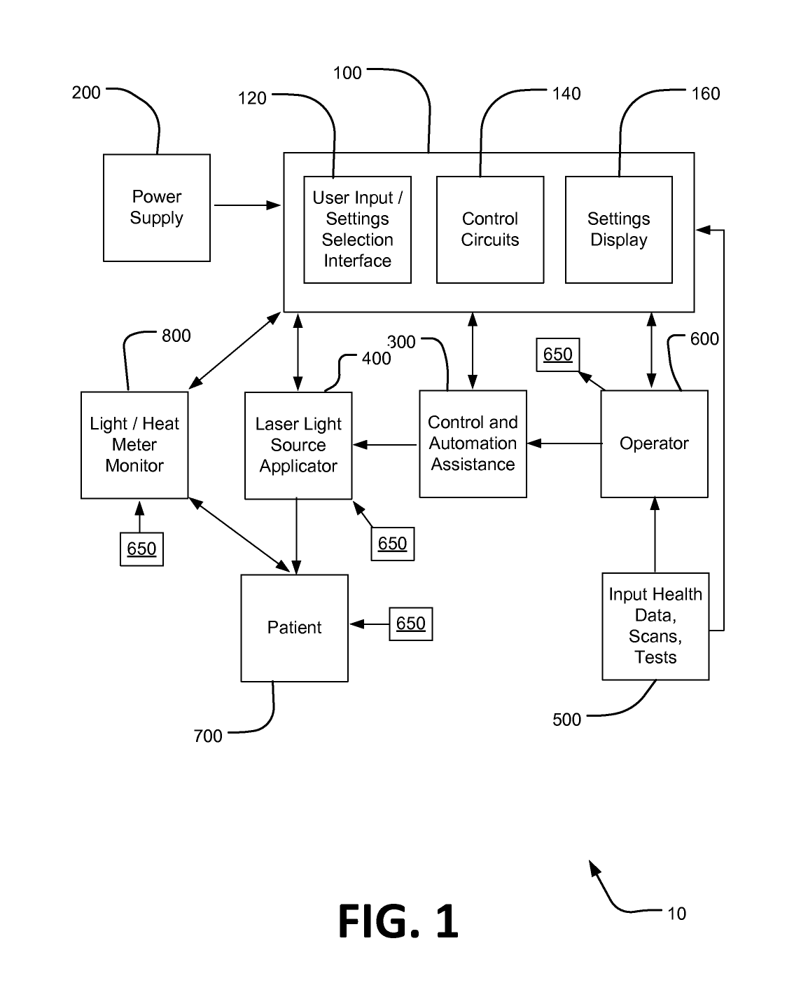

Referring now to the drawings, FIG. 1 illustrates an exemplary embodiment of a laser treatment system 10 that can be utilized with one or more novel treatment methodologies using light therapy. The laser control and interface unit 100 allows an operator 600 to interact with the system 10, input settings, observe the status of the unit and any ongoing procedure, etc. For example, it may be useful to set the system to output multiple wavelengths simultaneously, while being able to specify different wattages and pulsing settings for each wavelength. Subcomponents include a user input/settings selection interface 120, electronics/control circuits 140, and the settings display 160. In other embodiments one or more of the subcomponents could be relocated in other portions of the system 10 or otherwise distributed (for example, the settings display information could be displayed on a heads-up type portable display worn by the operator, on a hand-held portable electronic device such as a smart phone, projected on a nearby surface, etc.).

The user input/settings selection interface 120 allows an operator 600 to configure the system 10, specify desired system settings, input patient/procedure specific information/settings, and otherwise control the system 10 itself The settings display 160 communicates current settings, system variables, etc. to the operator 600 so that he or she is fully informed of the system state on a real-time basis. Additional information concerning the patient, procedure, environment, etc. can also be communicated via the settings display 160.

Control circuits 140 comprise the functional electronics of the system 10 that receive user inputs/settings, configure the system accordingly, and drive the settings display 160 component. Furthermore, the control circuits 140 receive power from the power supply 200 (which can be a battery or other portable source, wall plug-in, etc.) and control the output characteristics of the laser light source applicator 400.

The laser light source applicator 400 can comprise a single applicator or a plurality of applicators. The applicators can be attached to one another or they can be moved independently from each other in some embodiments.

Control and automation assistance 300 can be software and/or hardware that assist the operator 600 in operating the laser light source applicator 400. It can automate changes in the settings, assist in movement of the applicator 400, provide for safety measures, etc. In another embodiment, no control and automation assistance 300 is present, leaving the operator to control the system 10.

The laser light source applicator 400 receives power, instructions, information and input from the laser control and interface unit 100, the control and automation assistance 300 and the operator 600 in order to generate output light with the specific characteristics desired for the particular treatment methodologies being used for a given patient 700.

The operator 600 can customize the system 10 for the needs of a particular patient based on the input health data, scans, tests, etc. 500. This specific information (e.g, functional brain scans for brain treatment; neurodiagnostic testing for polyneuropathy, etc.) can help the operator and the system correctly employ the novel light therapy treatment methodologies for a particular patient 700 and his or her specific needs. A light/temperature meter monitor (e.g., laser thermometer, light meter) 800, alternatively including display and print-out capabilities 800, is used to monitor in real-time the levels of infrared light utilized as well as the patient.

The operator 600 directly interacts with the laser control and interface unit 100, the control and automation assistance 300 and other components represented by interactive 650. Interactive 650 links the operator 600 directly to the laser light source applicator 400, the light/temperature meter monitor 800, and the patient 700. FIG. 1 utilizes the interactive 650 links to represent direct interactions with these other components rather than over-complicating the figure with additional connection arrows between the operator 600 and these additional components.

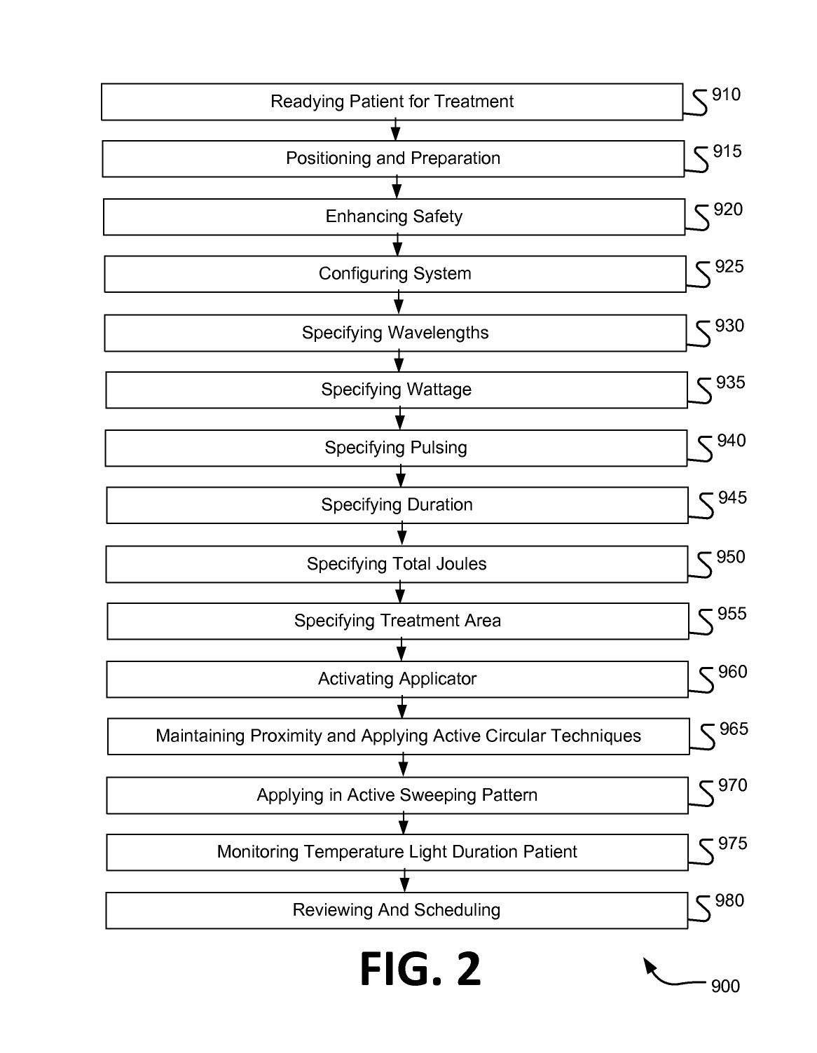

FIG. 2 illustrates a top plan view of exemplary operations of an embodiment of a novel treatment methodology using light therapy 900. The precise mechanisms underlying photobiomodulation and its therapeutic benefits are not fully understood. However, our research continues to build the knowledge-base in this area and shed light on the mechanisms and benefits of NIR as employed in our novel treatment methodologies using light therapy.

In the embodiment illustrated in FIG. 2, an exemplary novel treatment methodology using light therapy 900 is detailed in items 910-980. It should be understood by one skilled in the art that although some of the steps should occur before others, there is no strict requirement to the order of the steps and the invention may be practiced in various orders. Furthermore, although some of the steps are required, others are not, as delineated by the claims.

The step of Readying Patient for Treatment 910 involves a preliminary appointment with the patient wherein the patient is selected based on indications and absence of contraindications. The patient is informed of what to expect from the treatment, information about the laser itself, possible contraindications, alternate treatment options, etc. The patient can be asked to complete a questionnaire regarding medications, activities, allergies to infrared light, etc. Additionally, a patient may undergo diagnostic testing or imaging (SPECT or MRI) to aid in the localization of the area requiring treatment. A discussion of the area(s) to be specifically treated and why, based upon a SPECT scan or other diagnostic information, is conducted. The patient is also informed of the potential number of appointments to expect for treatments related to their condition; and any manufacturer's brochures and other information is distributed.

The step of Positioning and Preparation 915 involves preparing the patient for the treatment, including ensuring that no lotions or oils are present and removing or shortening hair in the region to be treated (cut to less than ½ inch or shaved), The patient is then positioned comfortably on a treatment table and the area to be treated is made fully accessible. Proper head support is important, especially when the head is to be treated.

The step of Enhancing Safety 920 involves ensuring that patient, operator, and anyone else in the room dons appropriate safety gear. For example, protective eyewear should be worn, which may include metal safety goggles, especially for the patient. The operator wears protective eyewear that can be customized to be specific to the wavelength(s) of the system.

The step of Configuring the System 925 involves setting the laser control and interface unit 100 to the appropriate specifications for a given condition and patient, which includes specifying the wavelength(s), wattage(s), continuous wave or pulse(s) and details of pulsing, duration of treatment (time), and total Joules to be delivered (area covered multiplied by time administered).

The step of Specifying Wavelengths 930 involves setting the system to output the desired wavelength(s) for treating a given condition and patient. Although only one particular wavelength may be desired in some instances, at other times, the operator may need to utilize two, three or more wavelengths to be delivered simultaneously, serially, etc. For example, an operator may choose to utilize multiple wavelengths (each potentially having distinct settings of pulsing and/or wattage, see below). The percentage activation of each wavelength can be variable; for example, an operator could select a 980 nm wavelength at 40% of time, and an 810 nm at 60% of the time.

The step of Specifying Wattage 935 involves setting the system to output the desired wattage(s) for treating a given condition and patient. Often, a single wattage is used throughout a particular treatment. However, the operator may choose to set the system to deliver varying wattages as needed. In many embodiments, the wattage is set between 10.01 and 20.00 watts. Generally, the operator selects the wattage based on desired tissue penetration, with higher wattages allowing deeper penetration. Because of blood flow in the tissues, temperatures at the surface and at depth generally normalize very quickly. Nevertheless, the system allows the operator to monitor temperatures and adjust the wattage, applicator motions, etc. as needed to ensure a safe temperature range (i.e., a skin temp at 99-102 degrees F.).

The step of Specifying Pulsing 940 involves setting the system to output the light in a non-continuous or pulsed mode. Pulsing may be omitted when not needed; alternatively, pulsing the infrared light can be utilized when indicated in the specific protocol. When the operator wishes to decrease the temperature of the treated area, he or she can vary the pulse rate; for example, choosing a longer rest period will cause the temperature to decrease. Alternatively, decreasing the therapeutic period can also cause the temperature to decrease. The operator can utilize the system to control how long either period of the pulse lasts. Pulsing of infrared light also increases the depth of penetration and the amount of energy delivered to any given point at the peak of a pulse. Yet, pulsing allows for troughs of energy output such that the overall energy delivered to the tissue can be equivalent or even lower than that delivered by a continuous emission.

The step of Specifying Duration 945 involves determining the length of time of a given treatment. The time varies depending on the area to be treated, patient parameters (e.g., the size of the head, which is mostly dependent on age and sex of individual; tissue bulk overlying a joint to be treated), and therapeutic response. The standard treatment time is usually less than 30 minutes, but can be longer as needed.

The step of Specifying Total Joules 950 involves determining the total Joules to be delivered during the treatment. This can be calculated based on wattage, pulsing, treatment duration, and area to be treated; adjustments can be made thereto in order to adjust the total Joules desired.

The step of Specifying Treatment Area 955 involves measuring the area to be treated and recording it in the chart.

The step of Activating Applicator 960 involves the operator actually activating the laser light and applying the applicator to the patient's skin (or in close proximity thereto, about zero to ten millimeters). In one embodiment, the laser light source applicator 400 comprises a single applicator; in another embodiment, it comprises a plurality of applicators. The applicators can be attached to one another or they can be moved independently from each other in some embodiments.

The step of Maintaining Proximity and Applying Active Circular Techniques 965 involves the operator maintaining the applicator in contact with the skin or in close proximity thereto; yet, at the same time, maintaining the applicator in continuous motion. A finger or applicator distance device can be used to assist the operator in maintaining the appropriate distance between the applicator and the skin. As the distance between the applicator and the patient's skin increases, the increasing amount of air that the laser must pass through causes a decrement in the power of the laser light. This loss of photonic energy significantly decreases the amount of tissue penetration and reduces the efficacy of the treatment. During application of the light, the operator can apply an active circular motion technique to keep the applicator moving in overlapping circles having approximately 50% overlap and moving linearly from one circle to the next. The distance covered in the overlapping circular motion depends on the size of the applicator; in one embodiment circles with diameters of approximately 2-3 centimeters can be used.

The step of Applying in Active Sweeping Pattern 970 involves the operator continuing the active circular techniques from 965 while moving linearly across (or down) the treatment area, followed by offsetting with a 50% overlap and sweeping back the other direction. This active sweeping pattern comprises a continuous sweeping motion that is maintained in order to minimize heat build-up and/or patient discomfort. The active sweeping pattern continues until the entire treatment area has been traversed. In some embodiments, multiple traversals of the treatment area are required.

The step of Monitoring Temperature Light Duration Patient 975 involves the operator and the system monitoring multiple items including the temperature of the patient's skin, the light being output, the duration of the treatment, and the patient. This is an ongoing step throughout the treatment. A laser thermometer and other monitoring devices can be integrated into the applicator or be separate components.

The step of Review and Scheduling 980 involves reviewing the treatment with the patient, interviewing the patient about his or her condition and experiences during the treatment and scheduling future treatments (as most conditions require multiple treatment sessions).

In one embodiment, novel treatment methodologies using light therapy can be employed to treat an exemplary case of polyneuropathy. In this example, a neurologist or other medical professional can conduct an EMG study (or other diagnostic procedure) to determine which nerve is involved, locate the distal portion of the nerve, etc. Then, after completing 910, 915 and 920, the operator configures the system. For example, by setting the wattage to 10.5 watts, setting the wavelengths to 810 nm and 980 nm, specifying continuous wave, setting duration at 10 minutes, and determining total joules to be about 6000. The operator would then activate the applicator over the nerve to be treated. Maintaining proximity and applying active circular techniques, the operator would move the applicator over the area of involvement (tracing the actual nerve/nerves, motor and sensory), from the knee to the foot (i.e., nerve tracing). The active sweeping pattern is maintained throughout, while monitoring the temperature, light, duration and patient as well. The treatment is then reviewed with the patient and follow-up treatments are scheduled (usually approximately 9-10 treatments for this condition). Data from past treatments as above have shown a 20-30% increase in the conduction velocity of the nerves involved.

In summary, during NIR phototherapy, absorption of red or NIR photons by COX in the mitochondrial respiratory chain causes secondary molecular and cellular events, including activation of second messenger pathways, changes in NO levels, and growth factor production. NILT leads to the reduction of excitotoxicity, the production of neurotrophic factors, the modulation of ROS, the transcription of new gene products with protective or pro-proliferative properties, and the release of numerous growth factors for neurons and other cells. NIR appears to initiate a cascade of subcellular events which can yield immediate, delayed, and persistent beneficial changes in the injured neuron or other cell.

Extensive research has shown that fluence within the range of 0.9-15.0 J/cm² is most effective in activating the biological processes involved in reversing or mitigating the pathophysiological effects of TBI. The attenuation of NIR energy as it passes through tissue has been examined in computer simulations, animal tissue, and human tissue. NIR penetration to the human brain is subject to attenuation by multiple tissues (skin, skull, dura, blood, cerebrospinal fluid) and multiple interfaces which scatter, absorb, and reflect the NIR light to varying degrees. We have shown through the use of higher wattage NIR lasers that we can deliver fluence at therapeutic levels to the depths of the brain without tissue heating or damage. The protocols have been applied in our clinic with excellent clinical results and no side effects.

Review of the neuroimaging literature on TBI has revealed that the most common areas injured in TBI are the orbitofrontal cortex (at the ventral surface of the frontal lobe) and the anterior and medial temporal lobes. It is not anatomically possible to position an NIR light emitter immediately exterior to the skull overlying these areas. Indeed, the orbitofrontal cortex positioned immediately above the eyes can only be reached from the forehead by angling the light emitter. Similarly, the temporal lobes are separated from the surface by epidermis, dermis, subcutaneous fat, subcutaneous blood vessels, accessory head of the temporalis muscle, connective tissue, temporalis muscle, skull, and dura mater. Each of these structures has different absorption and refraction properties, and each interface between different materials also creates a barrier to transmission of photonic energy. Blood flowing in the subcutaneous vessels is believed to create a unique barrier to transmission. In summary, effectively targeting the areas most commonly injured in TBI with sufficient photonic energy to initiate reparative processes represents a significant challenge in NILT. This appears to have been overcome with the high-power laser methodologies presented here.

One concern about high-watt NIR lasers is the risk of tissue heating. We explored this issue. Temperature change was one to three degrees C. at the skin surface using continuous-wave NIR lasers in the range of 10-15 W. Using pulsed settings, the high-powered lasers showed no significant temperature change in tissue samples. The temperature change on human skin was one degree C. or less in the in vivo penetration studies while maintaining continuous movement of the laser probe head. Clinically, patients in this case series reported only slight warming of the skin, but no discomfort, using the continuous motion technique.

In one embodiment, an exemplary protocol is as follows Infrared light in the wavelength range of 600-1200 nm of a power of 10.01-20.00 watts is applied to the skin over the area or structure to be treated. The laser emitter device is set to protocols specific to the condition and to the patient. Variations in the use of continuous or pulsed light, type and rate of pulsing, wavelengths, duration, and power density are included in this methodology. The patient is positioned to be comfortable and so that the area to be treated is fully accessible. The patient and the operator wear protective safety gear such as eyewear. If the area to be treated is the brain, then the hair on the overlying scalp must be cut to less than ½ inch or shaved. The operator applies the infrared light applicator directly to or very close to the skin of the patient. A continuous motion must be maintained to minimize heating and/or patient discomfort. A sweeping or scanning action also facilitates covering large areas to be treated. Dosing is dependent on area to be treated, wattage, pulsing, duration, and distance from the skin Infrared application is continued for the duration of treatment appropriate to the area/structure to be treated. Temperature is monitored by laser thermometer or other light/heat meter monitor device. Duration of treatment varies depending on area to be treated, patient parameters, and therapeutic response. For most conditions, multiple treatments are required. These treatments are conducted at intervals not less than every 48 hours. Protocols for TBI, IPD, ADSD, PTSD, DA, MS, SCI, PNL, RDL, STR, MTD and other diseases, conditions, and dysfunction are distinct and disease-specific. Conditions involving different areas of the body have distinct methodologies.

While particular embodiments have been described and disclosed in the present application, it is clear that any number of permutations, modifications, or embodiments may be made without departing from the spirit and the scope of this disclosure.

Particular terminology used when describing certain features or aspects of the embodiments should not be taken to imply that the terminology is being redefined herein to be restricted to any specific characteristics, features, or aspects with which that terminology is associated. In general, the terms used in the following claims should not be construed to be limited to the specific embodiments disclosed in the specification, unless the above Detailed Description section explicitly defines such terms. Accordingly, the actual scope of the claims encompasses not only the disclosed embodiments, but also all equivalent ways of practicing or implementing the claimed subject matter.

The above detailed description of the embodiments is not intended to be exhaustive or to limit the disclosure to the precise embodiment or form disclosed herein or to the particular fields of usage mentioned above. While specific embodiments and examples are described above for illustrative purposes, various equivalent modifications are possible within the scope of the disclosure, as those skilled in the relevant art will recognize. Also, the teachings of the embodiments provided herein can be applied to other systems, not necessarily the system described above. The elements and acts of the various embodiments described above can be combined to provide further embodiments.

Any patents, applications and other references that may be listed in accompanying or subsequent filing papers, are incorporated herein by reference. Aspects of embodiments can be modified, if necessary, to employ the systems, functions, and concepts of the various references to provide yet further embodiments.

In light of the above “Detailed Description,” the inventors may make changes to the disclosure. While the detailed description outlines possible embodiments and discloses the best mode contemplated, no matter how detailed the above appears in text, embodiments may be practiced in a myriad of ways. Thus, implementation details may vary considerably while still being encompassed by the spirit of the embodiments as disclosed by the inventors. As discussed herein, specific terminology used when describing certain features or aspects should not be taken to imply that the terminology is being redefined herein to be restricted to any specific characteristics, features, or aspects of the embodiments with which that terminology is associated.

While certain aspects are presented below in certain claim forms, the inventors contemplate the various aspects in any number of claim forms. Accordingly, the inventors reserve the right to add additional claims after filing the application to pursue such additional claim forms for other aspects.

The above specification, examples and data provide a description of the structure and use of exemplary implementations of the described systems, articles of manufacture and methods. It is important to note that many implementations can be made without departing from the spirit and scope of the disclosure.

What is claimed is:

1. A method of novel treatment using light therapy to treat at least one of a set of conditions including Traumatic Brain Injury, Idiopathic Parkinson's Disease, Alzheimer's Disease, Senility, Dementia, Post-Traumatic Stress Disorder, Depression, and Anxiety, the method comprising:
readying a patient for treatment by ensuring patient has at least one of the set of conditions and has no allergy to infrared light;
preparation and positioning the patient by ensuring that an area to be treated has no lotions and no oils present, that any hair present is cut short, and that the area to be treated is made fully accessible;
enhancing safety by ensuring that patient and operator wear safety gear;
configuring a system by setting a laser control and interface unit to appropriate specifications for a given condition and patient, which includes specifying a plurality of wavelengths between 600 and 1200 nanometers, specifying a plurality of wattages between 10.01 and 20.00 watts, and specifying whether a laser will pulse and details of any pulses;
activating applicator by turning on a flow of laser light and applying an applicator having a width to within a proximity of the patient's skin from zero to ten millimeters; and
maintaining proximity and applying active circular motion techniques by maintaining the proximity while actively moving the applicator across the skin.

2. The method of novel treatment using light therapy of claim 1, further comprising:
wherein the active circular motion techniques involve moving the applicator in a small circle having a diameter between one and two centimeters.

3. The method of novel treatment using light therapy of claim 1, further comprising:
applying in active sweeping pattern by continuing the active circular motion techniques while actively moving the applicator linearly in one of an across, up and down direction through the treatment area, followed by offsetting with an overlap and sweeping back an opposite direction in order to minimize heat build-up; and
continuing the active sweeping pattern until the entire treatment area has been traversed.

4. The method of novel treatment using light therapy of claim 2, further comprising:
applying in active sweeping pattern by continuing the active circular motion techniques while actively moving the applicator linearly in one of an across, up and down direction through the treatment area, followed by offsetting with an overlap and sweeping back an opposite direction in order to minimize heat build-up; and
continuing the active sweeping pattern until the entire treatment area has been traversed.

5. The method of novel treatment using light therapy of claim 1, further comprising:
monitoring temperature by sampling a temperature of the patient's skin in the area to be treated throughout a duration of treatment.

6. The method of novel treatment using light therapy of claim 2, further comprising:
monitoring temperature by sampling a temperature of the patient's skin in the area to be treated throughout a duration of treatment.

7. The method of novel treatment using light therapy of claim 3, further comprising:
monitoring temperature by sampling a temperature of the patient's skin in the area to be treated throughout a duration of treatment.

8. The method of novel treatment using light therapy of claim 4, further comprising:
monitoring temperature by sampling a temperature of the patient's skin in the area to be treated throughout a duration of treatment.

9. A method of novel treatment using light therapy to treat at least one of a set of conditions including Multiple sclerosis, Spinal Cord Injuries, Mild-to-Moderate Stroke, Neuropathy/Polyneuropathy, Radiculopathy/Radiculitis, and mitochondrial dysfunction, the method comprising:
readying a patient for treatment by ensuring patient has at least one of the set of conditions and has no allergy to infrared light;
preparation and positioning the patient by ensuring that an area to be treated has no lotions and no oils present, that any hair present is cut short, and that the area to be treated is made fully accessible;
enhancing safety by ensuring that patient and operator wear safety gear;
configuring a system by setting a laser control and interface unit to appropriate specifications for a given condition and patient, which includes specifying a plurality of wavelengths between 600 and 1200 nanometers, specifying a plurality of wattages between 10.01 and 20.00 watts, and specifying whether a laser will pulse and details of any pulses;
activating applicator by turning on a flow of laser light and applying an applicator having a width to within a proximity of the patient's skin from zero to ten millimeters; and
maintaining proximity and applying active circular motion techniques by maintaining the proximity while actively moving the applicator across the skin.

10. The method of novel treatment using light therapy of claim 9, further comprising:
wherein the active circular motion techniques involve moving the applicator in a small circle having a diameter between one and two centimeters.

11. The method of novel treatment using light therapy of claim 9, further comprising:
applying in active sweeping pattern by continuing the active circular motion techniques while actively moving the applicator linearly in one of an across, up and down direction through the treatment area, followed by offsetting with an overlap and sweeping back an opposite direction in order to minimize heat build-up; and
continuing the active sweeping pattern until the entire treatment area has been traversed.

12. The method of novel treatment using light therapy of claim 10, further comprising:
applying in active sweeping pattern by continuing the active circular motion techniques while actively moving the applicator linearly in one of an across, up and down direction through the treatment area, followed by offsetting with an overlap and sweeping back an opposite direction in order to minimize heat build-up; and
continuing the active sweeping pattern until the entire treatment area has been traversed.

13. The method of novel treatment using light therapy of claim 9, further comprising:
monitoring temperature by sampling a temperature of the patient's skin in the area to be treated throughout a duration of treatment.

14. The method of novel treatment using light therapy of claim 10, further comprising:
monitoring temperature by sampling a temperature of the patient's skin in the area to be treated throughout a duration of treatment.

15. The method of novel treatment using light therapy of claim 11, further comprising:
monitoring temperature by sampling a temperature of the patient's skin in the area to be treated throughout a duration of treatment.

16. The method of novel treatment using light therapy of claim 12, further comprising:
monitoring temperature by sampling a temperature of the patient's skin in the area to be treated throughout a duration of treatment.
